(19) United States
(12) Patent Application Publication
(10) Pub. No.: US 2025/0144142 A1
(43) Pub. Date: May 8, 2025

(54) METHODS OF PROTECTING IMPLANTED OR TRANSPLANTED CELLS AND TISSUES FROM IMMUNE REJECTION

(71) Applicants: MUSC FOUNDATION FOR RESEARCH DEVELOPMENT, Charleston, SC (US); CLEMSON UNIVERSITY, Clemson, SC (US)

(72) Inventors: Leonardo M.R. FERREIRA, Charleston, SC (US); Ying MEI, Mount Pleasant, SC (US); Dimitrios Chrisovalantou ARHONTOULIS, Charleston, SC (US); Jaime VALENTIN-QUIROGA, Madrid (ES)

(73) Assignees: MUSC FOUNDATION FOR RESEARCH DEVELOPMENT, Charleston, SC (US); CLEMSON UNIVERSITY, Clemson, SC (US)

(21) Appl. No.: 18/931,353

(22) Filed: Oct. 30, 2024

Related U.S. Application Data
(60) Provisional application No. 63/595,818, filed on Nov. 3, 2023.

(51) Int. Cl. A61K 35/17; A61K 39/00; A61K 40/11; A61K 40/31; A61K 40/42; C12N 5/0783; C12N 15/86

(52) U.S. Cl. CPC A61K 35/17; A61K 39/001112; A61K 40/11; A61K 40/31; A61K 40/4211; C12N 5/0637; C12N 15/86; C12N 2501/2302; C12N 2510/00; C12N 2740/15043

(57) ABSTRACT

The present disclosure is directed to method for the generation and use of regulatory T cells (Tregs) in the prevention and treatment of graft/transplant rejection. In particular, the method employs engineered CAR Treg cells that protect transplanted materials from rejection.

### PRIORITY CLAIM

This application claims benefit of priority to U.S. Provisional Application Ser. No. 63/595,818, filed Nov. 3, 2024, the entire contents of which are hereby incorporated by reference.

### STATEMENT REGARDING FEDERALLY SPONSORED RESEARCH

This invention was made with government support under grant no. 5R01HL133308-02 awarded by the National Institutes of Health. The government has certain rights in the invention.

### BACKGROUND

### 1. Field of the Disclosure

The present disclosure relates generally to the fields of medicine, transplant biology and immunology. More particularly, the disclosure relates to the generation and use of regulatory T cells (Tregs) in the prevention and treatment of graft/transplant rejection.

### 2. Background

Cell therapy offers the promise to cure many diseases caused by the loss of one or more specialized cell types. For instance, cardiovascular disease is the leading cause of death worldwide. For instance, cardiovascular disease is the leading cause of death worldwide. Due the limited regenerative capacity of the human heart, human pluripotent stems cell-derived cardiomyocytes (hPSC-CMs) have received significant attention due to their proven capacity to restore contractile function upon transplantation to injured hearts. Yet, the financial barrier to creating autologous hPSC-CMs for each patient has limited this approach to allogeneic hPSC-CMS, which require the use of immunosuppressive drugs that are associated with adverse side effects.

The inability to cross the allogeneic barrier for hPSC-CM implantation has curbed their translation potential. While hypoimmunogenic “universal” hPSCs have been created, CMs derived from these hPSCs poses the risk of creating tissues that cannot be surveilled by the immune system and eliminated in case of viral infection or cancer formation. Hence, it would be desirable to instead induce immune tolerance, i.e., unresponsiveness specifically towards transplanted tissues.

Regulatory T cells (Tregs) are a subset of T cells dedicated to limiting unwanted excessive immune responses. While clinical studies have shown that infusing patients with polyclonal Tregs is safe, immune tolerance induction will require antigen-specific Tregs. However, such antigen-specific T regs are vanishingly rare and difficult to isolate. In addition, an emerging concern is CAR Treg-mediated toxicity and inflammatory cytokine production. It is thus essential to find solutions to these challenges and thereby permit use of T regs to modulate immune responses to transplanted materials.

### SUMMARY

Thus, in accordance with the present disclosure, there is provided a method of protecting an implanted cell, tissue, organ or organoid from immune response in a recipient subject comprising administering to said recipient subject a chimeric antigen receptor (CAR) T regulatory cell (T_{reg}), wherein said CAR T_{reg} recognizes a natural or artificial antigen that (a) is not found in said implanted cell, tissue, organ or organoid and is (b) co-administered with said implanted cell, tissue, organ or organoid or found in proximity to said implanted cell, tissue, organ or organoid once implanted. The antigen may be co-administered to said recipient subject and is associated with a particle (microparticle or nanoparticle), a polymer, a cell, a synthetic/engineered cell, or a synthetic cell surrogate. Alternatively, the antigen is found in proximity to said implanted cell, tissue, organ or organoid and located on an extracellular matrix or cell found at a site of implantation in said recipient subject.

The CAR T_{reg} cells may be generated from peripheral blood mononuclear cells or generated from cells that are CD4+, CD25+, and CD127−, such as generated from naïve cells that are CD4+, CD25+, CD127−, CD45RA+, and CD62L+. The CAR T_{reg} cells may be generated by anti-CD3/CD28 stimulation in the presence of IL-2, followed by transduction with a virus encoding the CAR, such as a lentivirus, or may be generated by anti-CD3/CD28 stimulation in the presence of IL-2, followed by CRISPR/Cas9 targeting of the alpha constant region of the T cell receptor (TCR) locus (TRAC) combined with transduction with a non-integrating adeno-associated virus (AAV) encoding the CAR, including where the transduced cells are expanded in the presence of IL-2.

The CAR T_{reg} cells may retain expression of FOXP3 and HELIOS at levels indicative of T_{reg} cells. The subject may be a human or non-human mammal, such as a subject that suffers from a disease or disorder benefiting from transplant therapy. The disease or disorder may be cardiovascular disease, cancer, autoimmune disease (e.g., Type 1 diabetes, autoimmune carditis) or Parkinson's disease. The implanted cell, tissue, organ or organoid may comprise cardiovascular cells, pancreatic beta cells, or dopaminergic neuron progenitors. The artificial antigen may be a truncated CD19 antigen.

In another embodiment, there is provided a method of transplanting a cell, tissue, organ, or organoid into a subject comprising (a) transplanting a cell, tissue, organ, or organoid into a subject; and (b) introducing into the subject a chimeric antigen receptor (CAR) T regulatory cell (T_{reg}), wherein said CAR T_{reg} recognizes a natural or artificial antigen that (a) is not found in said implanted cell, tissue, organ or organoid and is (b) found in proximity to said implanted cell, tissue, organ or organoid once implanted. The subject may suffer from a disease or disorder benefiting from transplant therapy, such as cardiovascular disease, cancer, autoimmune disease (e.g., Type 1 diabetes, autoimmune myocarditis), or Parkinson's disease.

In yet another embodiment, there is provided a method of transplanting a cell, tissue, organ, or organoid into a subject comprising (a) transplanting a cell, tissue, organ or organoid into a subject; and (b) administering to the subject (i) a chimeric antigen receptor (CAR) T regulatory cell (T_{reg}), wherein said CAR T_{reg} recognizes a natural or artificial antigen that is not found in said implanted cell, tissue, organ or organoid; and (ii) said natural or artificial antigen, wherein said natural or artificial antigen co-administered with said CAR T_{reg} or implanted at or near the site at which said cell, tissue, organ, or organoid is implanted. The subject may suffer from a disease or disorder benefiting from transplant therapy, such as cardiovascular disease, cancer, autoimmune disease (e.g., Type 1 diabetes, autoimmune myocarditis), or Parkinson's disease.

Also provided is kit comprising (a) a chimeric antigen receptor (CAR) T regulatory cell (T_{reg}) and (b) a natural or artificial antigen recognized by said CAR T_{reg}, optionally wherein said antigen is associated with a particle (microparticle or nanoparticle), a polymer, a cell, a synthetic cell, or a synthetic cell surrogate. The kit may further comprise (c) a functional organoid, such as a cardiac organoid.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features, and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Cardiac organoids for heart repair. Cardiovascular disease is the leading cause of death worldwide, with a lower survival rate for heart failure (53%) than for breast cancer (90%). The adult human heart has negligible regenerative capacity. Hence, part of the solution to treat cardiovascular disease is to replenish the functional contractile heart tissue using exogenous sources.

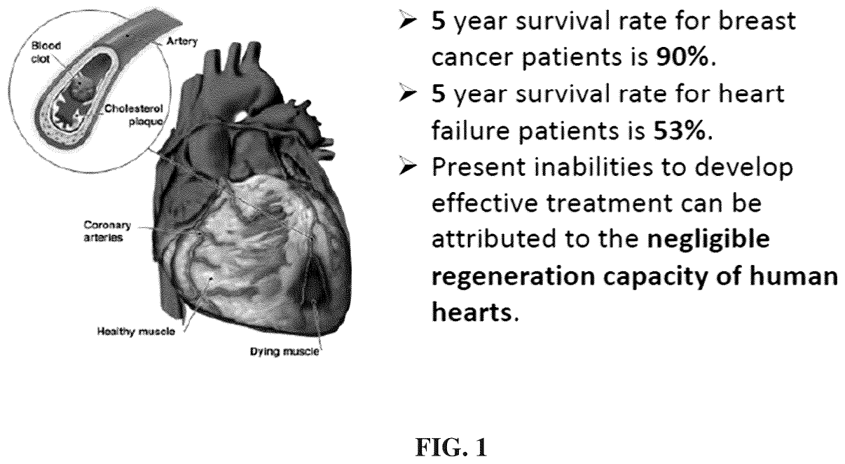

FIG. 2. Development of human cardiac organoids. Organoids are 3D microtissues based on the self-organization of stem cell-derived organ-specific cell types to recapitulate fundament tissue/organ structures and functions. Cardiac organoids are composed of human induced pluripotent stem cell-derived cardiomyoctyes (hiPSC-CM), cardiac fibroblasts (cFB), human vascular endothelial cells (HUVEC), and adipose-derived stems cells (hADSC). These cardiac organoids are spherical, beat, and stain positive for CD31 (endothelial cells), vimentin (fibroblasts), and alpha sarcomeric actinin (cardiomyocytes). DAPI stains the cells' nuclei.

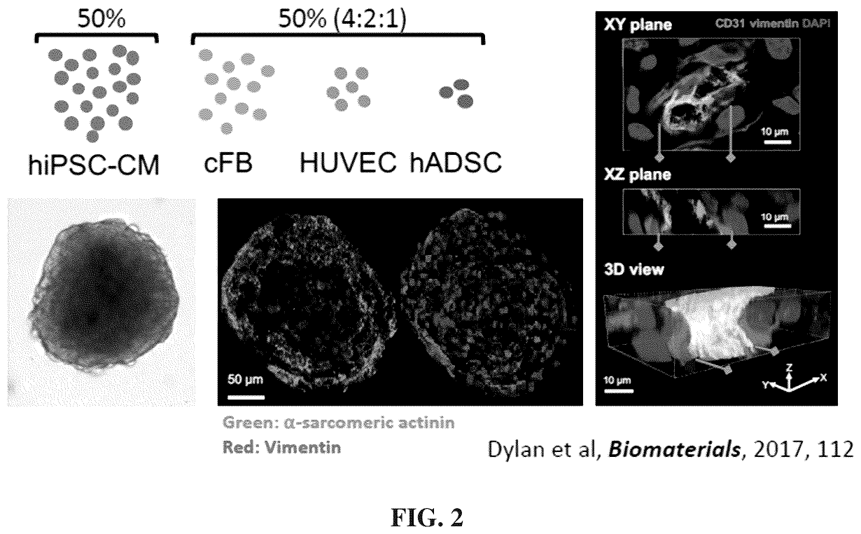

FIGS. 3A-E. Pre-vascularized, nanowired human cardiac organoids. These organoids better mimic normal heart embryonic development and stain positive for the same markers mentioned in FIG. 2, as well as Von Willebrand Factor (VWF) and the nanowires (NW) themselves.

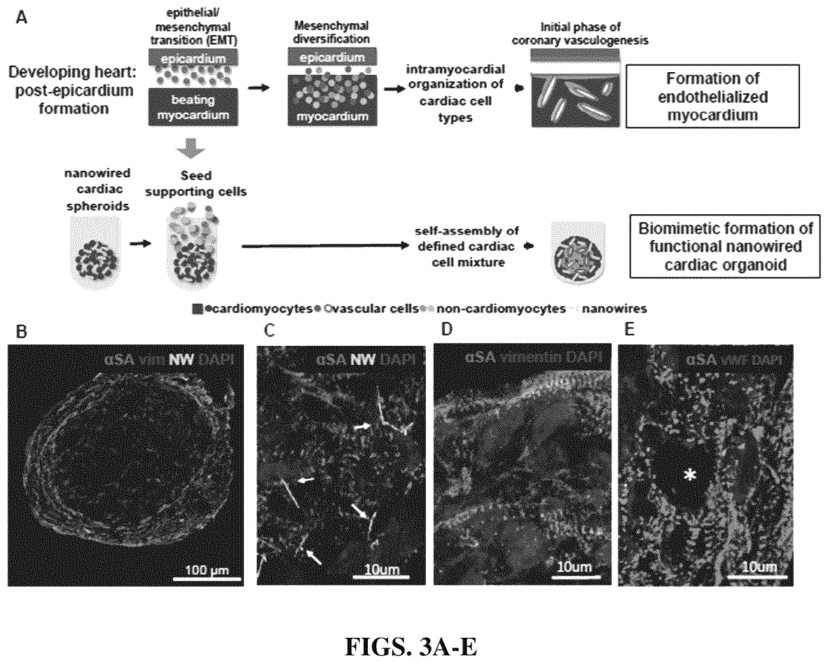

FIG. 4. Nanowired organoids improve functional recovery of ischemia/reperfusion injured rat hearts. Using 20-fold less hPSC-CMs (0.5 million/rat) as compared to state-of-the-art approaches, the inventors were able to achieve superior functional recovery. As can be seen across the three graphs in the bottom, nanowired cardiac organoids (far right bar in each set) led to better heart functional recovery as assessed by fractional shortening (FS) measurements.

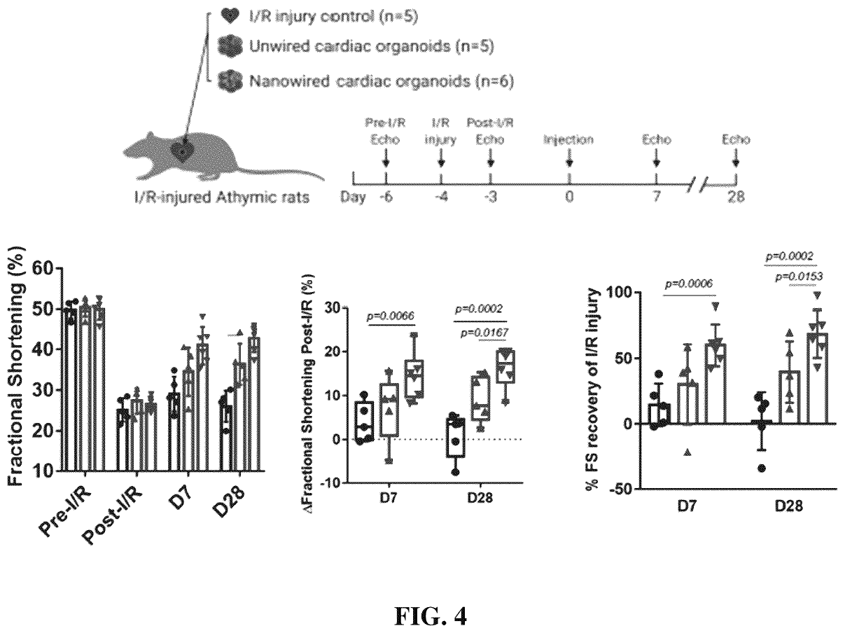

FIG. 5. Target chimeric antigen receptor regulatory T cells (CAR Tregs) to cardiomyocytes to protect cardiac organoids from immune rejection. Tregs are an immunosuppressive subset of T lymphocytes. Incorporating them into cardiac organoids can protect them from rejection by the immune recipient's immune cells. The inventors began by using Human Leukocyte Antigen A2 (HLA-A2)-expressing cardiac organoids and anti-HLA-A2 CAR Tregs.

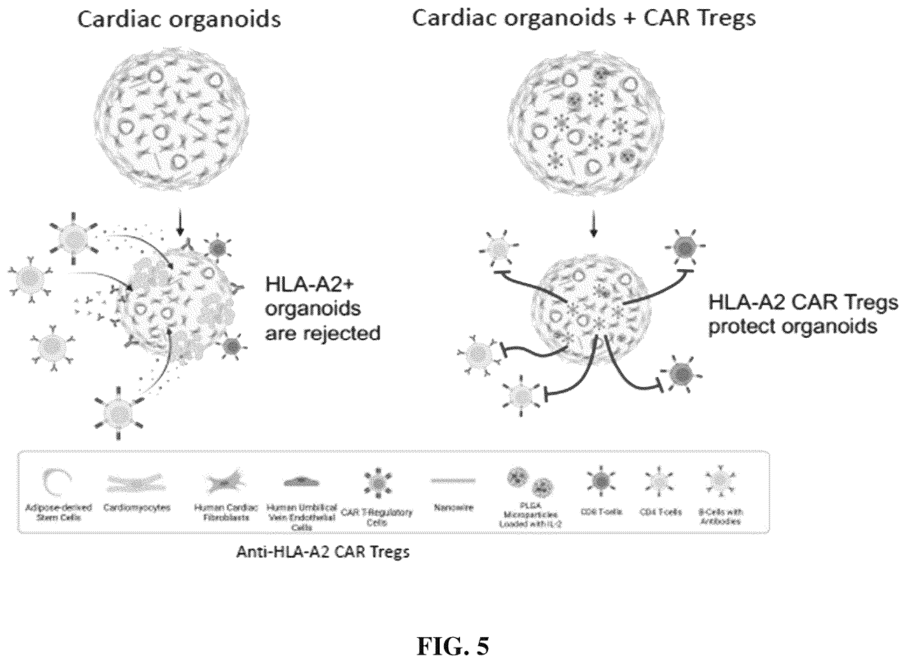

FIGS. 6A-H. Primary human Tregs were isolated from human peripheral blood by isolating peripheral blood mononuclear cells (PBMCs) from leukopaks, enriching CD4+ T cells by magnetic selection, and fluorescence-assisted cell sorting (FACS) for CD4+CD25+CD127− Tregs. In some instances, the inventors sorted CD4+CD25+CD127-CD45RA+CD62L+naïve Tregs. Tregs were sorted, activated with anti-CD3/CD28 beads and IL-2. Two days later, Tregs were transduced with lentivirus encoding the HLA-A2 CAR. Tregs were then expanded in the presence of IL-2 for an additional week until being used in experiments. As expected, HLA-A2 CAR Tregs were activated by HLA-A2-expressing irradiated K562 target cells but not by parental irradiated K562 cells, as measured by surface expression of the activation marker CD69 by flow cytometry (FIG. 6A). HLA-A2 CAR Tregs, but not untransduced (UT) Tregs, suppressed the proliferation of CellTrace Violet (CTV)-labeled anti-CD3/CD28-activated CD4+ T cells (FIG. 6B) and CD8+ T cells (FIG. 6C) upon activation by HLA-A2-expressing irradiated K562 target cells. Importantly, HLA-A2 CAR Tregs maintained their Treg phenotype, as evidenced by high expression of the Treg lineage transcription factors FOXP3 and HELIOS (FIG. 6D). However, HLA-A2 CAR Tregs, but no UT Tregs, co-incubated with HLA-A2+ cardiomyocytes were cytotoxic towards the cardiomyocytes, as assessed by lactate dehydrogenase (LDH) release (FIG. 6E). Similarly, HLA-A2 CAR Tregs destroyed HLA-A2+ cardiac organoid structure as assessed by immunofluorescence (FIG. 6F) and quantification of cardiac organoid beating (FIG. 6G). Interestingly, HLA-A2 CAR Tregs secreted high levels of TNF-alpha in the presence of HLA-A2+ cardiac organoids.

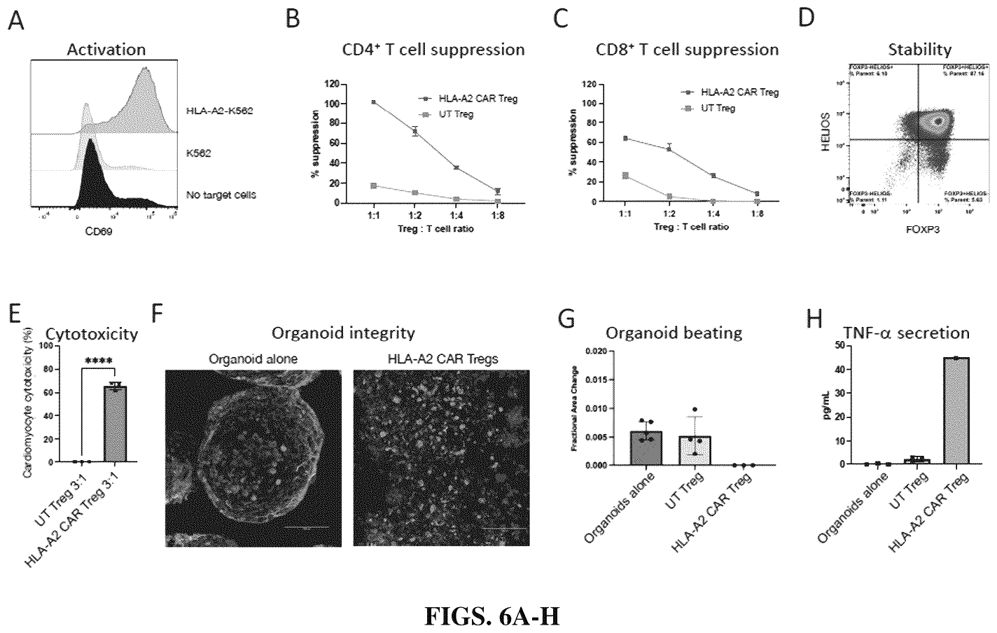

FIG. 7. HLA-A2 CAR Tregs are cytotoxic towards HLA-A2+ cardiomyocytes from two different sources, either stem cell-derived (left) or purchased from iCell (right).

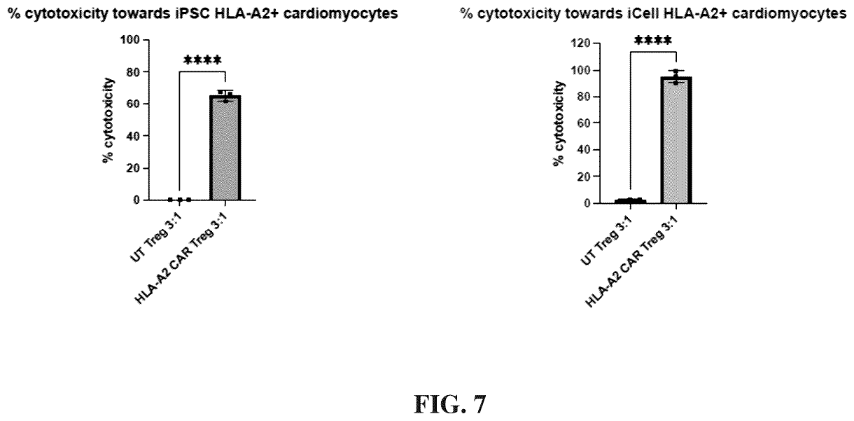

FIG. 8. HLA-A2 CAR Tregs also kill HLA-A2+ fibroblasts in monolayer (2D; left) and in spheroids (3D; right).

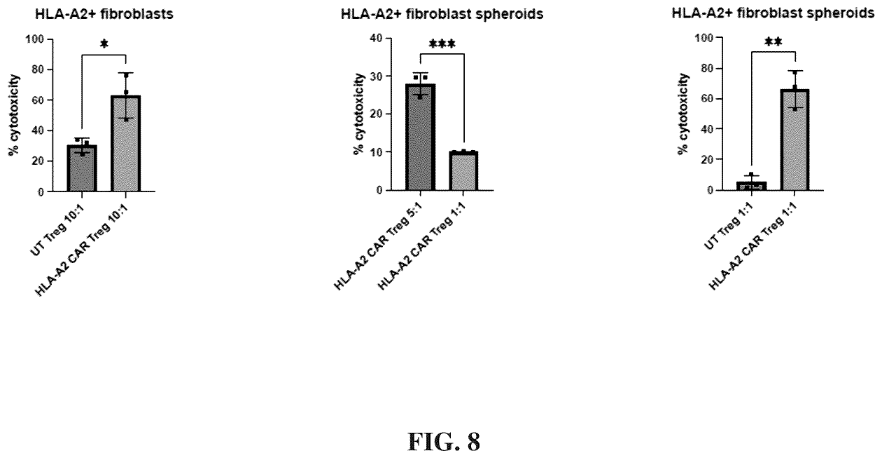

FIG. 9. CAR Tregs incorporated into cardiac organoids but targeted to an antigen that is not expressed on the cells of interest to avoid damage of the organoids. The inventors started by using CD19 CAR Tregs and irradiated CD19-expressing K562 cells as the source of antigen for the CD19 CAR Tregs.

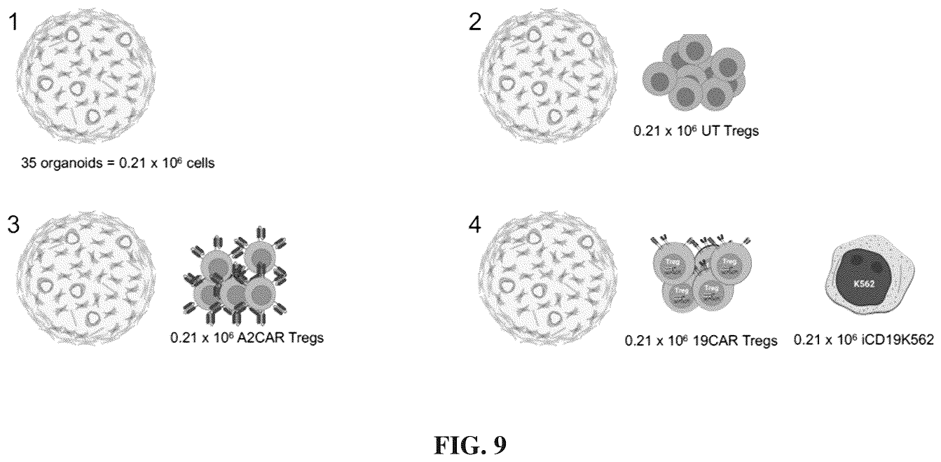

FIG. 10. Both HLA-A2 CAR Tregs incubated with HLA-A2+ cardiac organoids (A) and CD19 CAR Tregs incubated with HLA-A2+ cardiac organoids and irradiated CD19-expressing K562 cells (B) were activated, as assessed by CD69 surface expression. Yet, while HLA-A2 CAR Tregs damaged the cardiac organoids, as assessed by cardiac organoid beating (C) and cytotoxicity (D), CD19 CAR Tregs did not. In panel E one can see images of each condition's organoids.

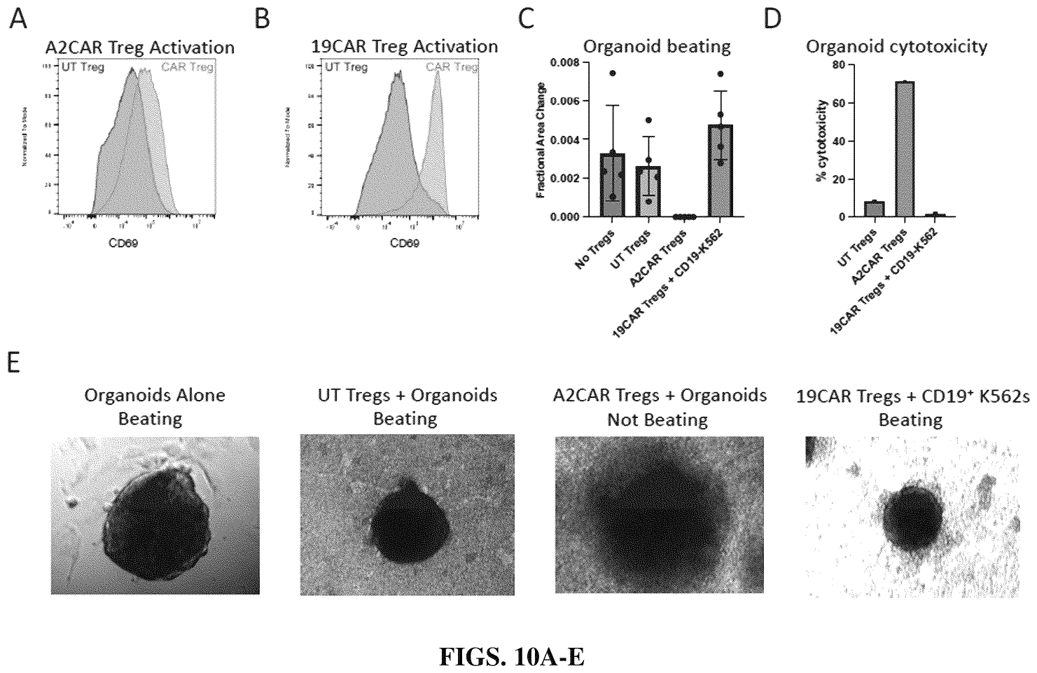

FIG. 11. Target CAR Tregs to microparticles in cardiac organoids to protect cardiac organoids from immune rejection (indirect targeting). In data collected so far involving cardiac organoids, the inventors used irradiated CD19-K562 as an approximation of microparticles, as irradiated K562 cells do not divide. This strategy allows for CAR Treg-mediated protection of tissues and organs to be transplanted without risking damage to said tissues and organs, an important step up from current strategies which are yet to be clinically demonstrated, involving engineered Tregs.

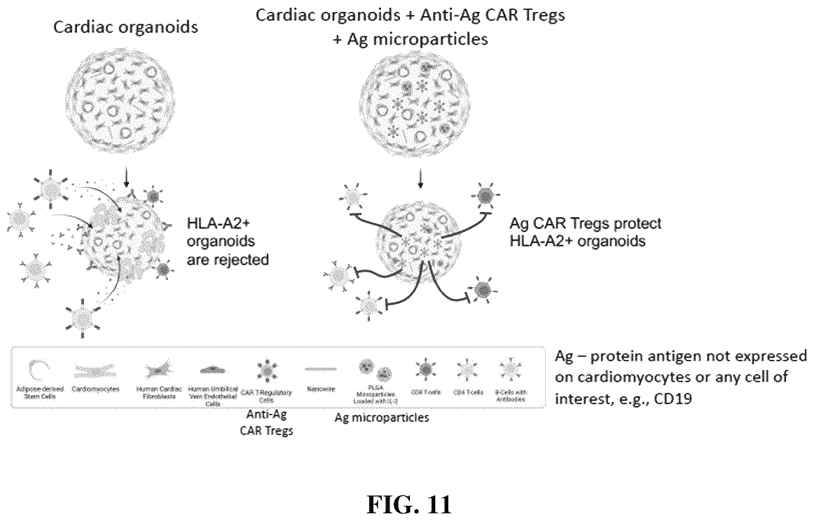

FIG. 12. CD19-coated beads are effective in specifically activating CD19 CAR Tregs. CD19 CAR (A) or untransduced (B) Tregs were cultured alone or with anti-CD3/CD28 beads, irradiated CD19-K562 cells (iCD19K562), or CD19-coated beads. Cell surface expression levels of the T cell activation marker CD71 were determined 48h later by flow cytometry. All bead to cell and cell to cell ratios were 1:1. UT, untransduced.

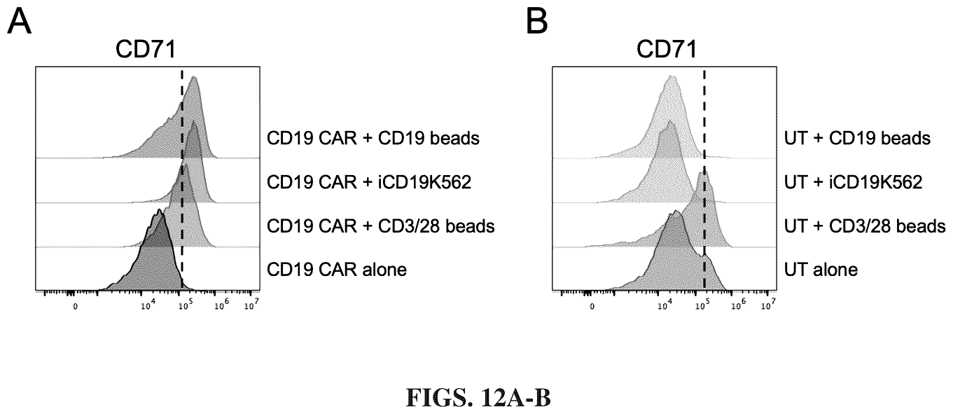

### DESCRIPTION OF ILLUSTRATIVE EMBODIMENTS

As discussed above, Tregs have the potential to reduce or eliminate unwanted host immune responses in transplant settings, yet have not reached their potential due to the challenge in identifying and isolating proper antigen-specific Tregs, as well as concerns over CAR Treg -mediated toxicity and inflammatory cytokine production.

Here, the inventors have found that CAR Tregs that directly target an antigen expressed on the tissue to be protected from immune rejection can result in the destruction and loss of that tissue. This destruction has been attributed to direct cell-cell interactions between CAR Tregs and the implanted cells and thus such a strategy, which is intended to protect the transplant, can actually cause cytotoxicity against the very tissue the CAR Tregs were designed to protect.

The inventors therefore developed a solution that they refer to as “indirect targeting CAR Treg.” Instead of using a CAR Treg that directly targets an antigen expressed on the transplanted tissue, they use CAR Tregs that target an antigen not found in/on the tissue of interest. Rather, the CAR Treg targets an antigen that exists nearby or is delivered in conjunction with the transplanted tissue. The antigen may be a naturally occurring antigen located in tissue or a tissue matrix that is located near a transplant site or may be artificial and/or located on a material co-transplanted with the tissue of interest.

These and other aspects of the disclosure are described in detail below.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” means, in general, within a standard deviation of the stated value as determined using a standard analytical technique for measuring the stated value. The terms can also be used by referring to plus or minus 5% of the stated value.

The phrase “effective amount” or “therapeutically effective” means a dosage of a drug or agent sufficient to produce a desired result. The desired result can be subjective or objective improvement in the recipient of the dosage, increased lung growth, increased lung repair, reduced tissue edema, increased DNA repair, decreased apoptosis, a decrease in tumor size, a decrease in the rate of growth of cancer cells, a decrease in metastasis, or any combination of the above.

As used herein, the term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. The antibody may be a bi-specific antibody. In exemplary embodiments, antibodies used with the methods and compositions described herein are derivatives of the IgG class. The term antibody also refers to antigen-binding antibody fragments. Examples of such antibody fragments include, but are not limited to, Fab, Fabÿ, F(abÿ)2, scFv, Fv, dsFv diabody, and Fd fragments. Antibody fragments may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 10 amino acids and more typically will comprise at least about 200 amino acids. “Subject” and “patient” refer to either a human or non-human, such as primates, mammals, and vertebrates. In particular embodiments, the subject is a human.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

The term “T cell” refers to T lymphocytes as defined in the art and is intended to include thymocytes, immature T lymphocytes, mature T lymphocytes, resting T lymphocytes, or activated T lymphocytes. The T cells can be CD4⁺ T cells, CD8⁺ T cells, CD4⁺CD8⁺ T cells, or CD4⁻CD8⁻ cells. The T cells can also be T helper cells, such as T helper 1 (TH1), or T helper 2 (TH2) cells, or TH17 cells, as well as cytotoxic T cells, regulatory T cells, natural killer T cells, naïve T cells, memory T cells, or gamma delta T cells. T cells that differ from each other by at least one marker, such as CD4, are referred to herein as “subsets” of T cells.

“Regulatory T cells” or “Tregs” refer to a subset of T cells which act to suppress immune responses, thereby maintaining homeostasis and self-tolerance. Self-tolerance refers to a state of immune unresponsiveness towards self-antigens, important to avoid the development of autoimmune disease.

The term “chimeric antigen receptors (CARs),” as used herein, may refer to artificial T cell receptors, chimeric T cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins.

### II. CAR REGULATORY T CELLS

In certain embodiments, the present disclosure provides T_{regs} engineered to express a CAR vector. The CAR T_{regs} may be used to treat a disease or disorder, such as an autoimmune disease. Certain embodiments of the present disclosure concern obtaining a starting population of T_{regs}, modifying the T_{regs}, and administering the modified T_{regs} to a subject as protection against immune rejection with transplantation. In particular, the T_{regs} express CAR.

Regulatory T cells (T_{regs}), formerly known as suppressor T cells, are a subpopulation of T cells that modulate the immune system, maintain tolerance to self-antigens, and prevent autoimmune disease. T_{reg} cells are immunosuppressive and generally suppress or downregulate induction and proliferation of effector T cells. T_{reg} cells express the biomarkers CD4, FOXP3, and CD25 and are thought to be derived from the same lineage as naïve CD4⁺ cells. Because effector T cells also express CD4 and CD25, T_{reg} cells are very difficult to effectively discern from effector CD4⁺, making them difficult to study. Research has found that the cytokine transforming growth factor beta (TGF-β) is essential for T_{reg} cells to differentiate from naïve CD4⁺ cells and is important in maintaining T_{reg} cell homeostasis.

Mouse models have suggested that modulation of T_{reg} cells can treat autoimmune disease and cancer and can facilitate organ transplantation and wound healing. Their implications for cancer are complicated. T_{reg} cells tend to be upregulated in individuals with cancer, and they seem to be recruited to the site of many tumors. Studies in both humans and animal models have implicated that high numbers of T_{reg} cells in the tumor microenvironment is indicative of a poor prognosis, and T_{reg} cells are thought to suppress tumor immunity, thus hindering the body's innate ability to control the growth of cancerous cells. Immunotherapy research is studying how regulation of T cells could possibly be utilized in the treatment of cancer.

Chimeric antigen receptors (CARs)—also known as chimeric immunoreceptors, chimeric T cell receptors or artificial T cell receptors—are receptor proteins that have been engineered to give T cells the new ability to target a specific antigen. The receptors are chimeric in that they combine both antigen-binding and T cell activating functions into a single receptor. CAR T cell therapy uses T cells engineered with CARs to treat various diseases such as cancer. The premise of CAR-T onco-immunotherapy is to modify T cells to recognize cancer cells in order to more effectively target and destroy them. In this case, the are targeted to antigen that will trigger the Tregs to act in an immunosuppressive fashion in a local environment where a transplanted tissue is located.

CAR T cells can be derived either from T cells in a patient's own blood (autologous) or from the T cells of another, healthy, donor (allogeneic). Once isolated from a person, these T cells are genetically engineered to express a specific CAR, using a vector derived from an engineered lentivirus such as HIV. In some embodiments, the starting T cell population for creating CAR T_{regs} is derived from the blood, cord blood, bone marrow, lymph, or lymphoid organs, such as the thymus. In some aspects, the cells are human cells. The cells typically are primary cells, such as those isolated directly from a subject and/or isolated from a subject and frozen. With reference to the subject to be treated, the cells may be allogeneic and/or autologous. In some embodiments, the methods include isolating cells from the subject, preparing, processing, culturing, and/or engineering them, as described herein, and re-introducing them into the same patient, before or after cryopreservation.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., Tregs) but are present or expressed at relatively higher levels on certain other populations of Tregs. In some embodiments, regulatory T cells are separated from a peripheral blood mononuclear cell (PBMC) sample by negative selection of markers expressed on non-T cells, such as B cells, monocytes, or other white blood cells, such as CD14. In some aspects, CD4+CD25+CD127-Tregs are purified from human peripheral blood.

In some embodiments, the Tregs are autologous Treg cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days. For example, the cells may be cultured from 5 days, 5.5 days, or 5.8 days to 21 days, 21.5 days, or 21.8 days, such as from 10 days, 10.5 days, or 10.8 days to 14 days, 14.5 days, or 14.8 days.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. More specifically, rapid expansion provides an increase of at least about 200-fold (e.g., 200-, 300-, 400-, 500-, 600-, 700-, 800-, 900-, or greater) over a period of about 10 to about 14 days. Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15). The non-specific T-cell receptor (TCR) stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as a human leukocyte antigen A2 (HLA-A2) binding peptide, in the presence of a T-cell growth factor, such as 300 IU/ml IL-2. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2⁺ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, Maraba virus vectors, and group B adenovirus Enadenotucirev vectors.

In some embodiments, the CAR contains an extracellular antigen-recognition domain that specifically binds to an antigen. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061, WO2023019144A1, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3 (4): 388-398; Davila et al. (2013) PLOS ONE 8 (4): e61338; Turtle et al., Curr. Opin. Immunol., 2012 October; 24 (5): 633-39; Wu et al., Cancer, 2012 Mar. 18 (2): 160-75. In some aspects, the genetically engineered antigen receptors include a CAR as described in U.S. Pat. No. 7,446,190, and those described in International Patent Application Publication No.: WO/2014055668 A1.

In some embodiments, the CAR comprises: a) an intracellular signaling domain, b) a transmembrane domain, and c) an extracellular domain comprising an antigen binding region.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the invention includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, and 4-1BB (CD137). In addition to a primary signal initiated by CD35, an additional signal provided by a human costimulatory receptor inserted in a human CAR is important for full activation of NK cells and could help improve in vivo persistence and the therapeutic success of the adoptive immunotherapy.

In some embodiments, CAR is constructed with a specificity for a particular antigen (or marker or ligand), such as an antigen expressed or present in a particular cell type/extracellular material, or an artificial antigen that is a modified form of a natural antigen of one that is entirely synthetic. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a cell surface protein, particularly an inert cell surface protein. Antigens include carbohydrate antigens recognized by pattern-recognition receptors, such as Dectin-1.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR⁺ immune cells (Singh et al., 2008; Singh et al., 2011).

### III. FORMULATION AND ADMINISTRATION

The present disclosure provides pharmaceutical compositions comprising PSC-derived cells and CAR Tregs. Such compositions comprise a prophylactically or therapeutically effective amount of Tregs and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Generally, the ingredients of the compositions of the disclosure are supplied either separately or mixed together in unit dosage form, for example, as a dry lyophilized powder or water-free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### IV. COMBINATION THERAPIES

In certain embodiments, the compositions and methods of the present embodiments involving Tregs administration are combined with a second or additional therapy. In certain embodiments, the compositions and methods of the present embodiments involve administering Tregs and at least one therapy for preventing immune rejection. Immunosuppressive agents for preventing immune rejection are well known in the field of transplant biology and include Prednisone, Tacrolimus (Prograf), Cyclosporine (Neoral), Mycophenolate Mofetil (CellCept), Imuran (Azathioprine), and Rapamune (Rapamycin, Sirolimus).

Combination therapies may enhance the therapeutic or protective effect, and/or increase the therapeutic effect of another therapy, thereby permitting lower doses of one or both. Therapeutic and prophylactic methods and compositions can be provided in a combined amount effective to achieve the desired effect. This process may involve contacting the cells with both an antibody or antibody fragment and a second therapy. A tissue, tumor, or cell can be contacted with one or more compositions or pharmacological formulation(s) comprising one or more of the agents, or by contacting the tissue, tumor, and/or cell with two or more distinct compositions or formulations.

In certain embodiments, a course of treatment will last 1-90 days or more (this range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this range includes intervening days), depending on the condition of the patient, such as their prognosis, strength, health, etc. It is expected that the treatment cycles are repeated as necessary.

### V. KITS

In various aspects of the embodiments, a kit is envisioned containing therapeutic agents and/or other therapeutic and delivery agents. In some embodiments, the present embodiments contemplate a kit for preparing and/or administering a Treg cell composition of the embodiments. The kit may comprise one or more sealed vials containing any of the pharmaceutical compositions of the present embodiments. The kit may include, for example, Treg cells as well as reagents to prepare, formulate, and/or administer the components of the embodiments or perform one or more steps of the inventive methods. In some embodiments, the kit may also comprise a suitable container, which is a container that will not react with components of the kit, such as an Eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Cardiac organoid manufacture: HLA-A2+ hPSC-CMs (CDI, Madison, WI) were used to fabricate HLA-A2+ human cardiac organoids with hcFBs, HUVECs, and hADSCs (Lonza, Basel, Switzerland), as recently published by us. Cardiac organoid structure and beating were assessed.

CAR T_{reg} manufacture: Primary human regulatory T cells (T_{regs}) were obtained by isolating human peripheral blood mononuclear cells (PBMCs) from fresh HLA-A2 negative donor leukopaks, magnetically enriching for CD4⁺ cells, and then sorting CD4⁺CD25⁺CD127⁻ Tregs using fluorescence-assisted cell sorting (FACS). In parallel, bulk CD4⁺ T cells and CD8⁺ T cells were magnetically purified from the same PBMCs as effector T cell controls. Isolated cells were activated with anti-CD3/CD28 beads and human recombinant IL-2 at a concentration of 1,000 IU/ml for Tregs, 100 IU/ml for bulk CD4⁺ T cells, and 300 IU/ml for CD8⁺ T cells, and, two days later, transduced with lentivirus containing our previously described anti-HLA-A2 CD28-CD3zeta chimeric antigen receptor (CAR) or anti-CD19 CD28-CD3zeta CAR coupled to a green fluorescent protein (GFP) reporter gene by a 2A peptide. CAR Tregs were expanded for 7-10 days, sorted to purity using FACS based on GFP expression, and used for in vitro assays.

CAR Treg phenotype characterization: CAR Tregs were surface stained for Myc-tag to assess CAR surface expression in parallel with total expression (GFP expression), as well as intracellularly stained for FOXP3 and HELIOS to assess Treg phenotype and stability. CAR Tregs were co-incubated with either irradiated CAR antigen expressing-K562 cells (a myelogenous leukemia cell line devoid of other HLA molecules and CD80/86 costimulatory molecule expression) or cardiac organoids and 48h later surface expression levels of the activation marker CD71 were assessed on CAR Tregs, as well as the levels of cytokines in the cell culture medium.

CAR Treg immunosuppressive properties: In vitro suppressive function of CAR Tregs was assessed by co-incubating CAR Tregs with irradiated CAR antigen expressing-K562 cells and, in parallel, incubating Cell Trace Violet (CTV) labeled bulk CD4⁺ and CD8⁺ T cells at a 1:1 ratio with anti-CD3/CD28 beads overnight. The following day, activated bulk CD4⁺ and CD8⁺ T cells were debeaded and co-incubated with activated CAR Tregs at different CAR Treg: bulk T cell ratios. Suppression was measured based on inhibition of CTV-labeled bulk T cell proliferation. Unmodified Tregs (UT—Untransduced) were used as controls.

CAR Treg cytotoxic properties: In vitro cytotoxic function of CAR Tregs was assessed by co-incubating CAR Tregs with cardiomyocytes, fibroblasts, or cardiac organoids and, after 48h, measuring lactate dehydrogenase (LDH) release into the cell culture medium to gauge cell death.

### Example 2—Results

Cardiovascular disease causes 1 out of 4 deaths in the United States. Due to the limited regenerative capacity of the adult human heart, human pluripotent stem cell-derived cardiomyocytes (hPSC-CMs) have emerged as a powerful cellular source for cardiac repair and are proven to restore contractile function in injured hearts of numerous mammals, including rodents and non-human primates. Furthermore, clinical trials are underway for hPSC-CM transplantation in humans. To improve on this regenerative medicine approach to cardiovascular disease, the inventors developed prevascularized human cardiac organoids composed of hPSC-CMs, human cardiac fibroblasts, endothelial cells, and stromal cells. The inventors' prevascularized organoids (10⁶ cells/rat) robustly engraft in ischemia/reperfusion (IR) injured rat hearts (30% 1-week post-implantation) and lead to functional recovery to the same extent (40%) as previous studies using 10⁷ hPSC-CMs per rat.

Despite progress in improving hPSC-CM engraftment and functional recovery of infarcted hearts, the current inability to cross the allogeneic barrier for hPSC-CM implantation has greatly limited its translational potential. Generating cardiac organoids with autologous cells for every patient would be costly, laborious, and not scalable. However, using non-autologous cells would require life-long immunosuppression to curb immune rejection, the current standard in organ transplant. Regulatory T cells (Tregs) are a T cell subset dedicated to maintaining immune homeostasis by inhibiting specific immune responses, minimizing healthy tissue damage. Manipulating human Tregs offers the opportunity to induce immune tolerance, i.e., unresponsiveness towards a specific antigen, in the clinic. Infusing patients with up to 3 billion polyclonal Tregs is safe. Yet, polyclonal Tregs are unlikely to provide the ultimate solution to transplanted tissue rejection, as only a small percentage of the infused Tregs recognize and get activated by relevant antigens. Antigen-specific Tregs are significantly more potent suppressors than their polyclonal counterparts, being able to prevent and even reverse disease in mouse models of transplant rejection and autoimmunity. However, antigen-specific Tregs are exceedingly rare. To circumvent this hurdle, the inventors propose using chimeric antigen receptor (CAR) technology to engineer antigen-specific CAR Tregs to protect human cardiac organoid implants. A CAR consists of an extracellular antigen-binding domain linked to an intracellular signaling domain, driving T cell activation upon target recognition.

With the intention to protect HLA-A2+ cardiac organoids from immune attack, the inventors generated human anti-HLA-A2 CAR Tregs. These cells were activated and suppressed T cell proliferation in vitro while maintaining a stable Treg phenotype. Strikingly, however, HLA-A2 CAR Tregs were cytotoxic towards HLA-A2+ hPSC-CMs and HLA-A2+ cardiac organoids. Indeed, HLA-A2 CAR Tregs visibly disrupted cardiac organoid morphology and abrogated their ability to contract. Of note, HLA-A2 CAR Tregs secreted high amounts of the pro-inflammatory cytokine TNF-α in cardiac organoids. Treg-mediated cytotoxicity has been previously reported in the context of Tregs eliminating antigen-presenting cells (APCs) as a mechanism to dampen immune responses. Of note, Tregs are restricted to recognizing epitopes presented by HLA class II molecules, which are expressed almost exclusively in professional antigen presenting cells (APCs). Treg-mediated killing of non-immune cells is thus a problem created only by engineering Tregs with CARs recognizing antigens on non-immune cells such as HLA-A2, an HLA class I molecule expressed in all cell types of HLA-A2+ individuals.

To circumvent the problem of CAR Treg-mediated cytotoxicity, the inventors took advantage of one of the basic tenets of Treg function, bystander suppression, which refers to Tregs' capacity to suppress immune responses towards an antigen distinct from the one Tregs recognize as long as the Tregs and effector immune cells are in the same milieu. Specifically, the inventors generated human anti-CD19 CAR Tregs and incorporated them in human HLA-A2+ cardiac organoids together with irradiated CD19-expressing K562 cells, a cell line devoid of HLA or co-stimulatory molecules. CD19 is an antigen expressed specifically by B cells, absent in cardiac organoid cells. Similar to HLA-A2 CAR Tregs, CD19 CAR Tregs were activated within cardiac organoids. Yet, CD19 CAR T_{regs} did not compromise cardiac organoid function or viability.

### Example 3—Discussion

The inventors' strategy of utilizing indirect targeting CAR Tregs holds great promise to protect cardiac organoids from allogeneic rejection with engineered Tregs by locally activating and maintaining suppressive Tregs without risking insults to the cardiac organoids. Their work provides proof-of-concept demonstration of a method to activate and maintain CAR Tregs locally in human cardiac organoids to ultimately induce long-term protection from allogeneic immune rejection in allogeneic recipients without immunosuppressive drugs, laying the foundation to applying the emerging CAR Treg technology to cardiac regenerative medicine. Moreover, the knowledge developed here will accelerate the clinical applications of allogeneic engineered tissue transplantation to treat other devastating diseases, such as type 1 diabetes.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
WO200014257
WO2013126726
WO2012129514
WO2014031687
WO2013/166321
WO2013/071154
WO2013/123061
WO2023019144A1
U.S. Patent Publication US2002131960
U.S. Patent Publication US2013287748
U.S. Patent Publication US20130149337
U.S. Pat. No. 6,451,995
U.S. Pat. No. 7,446,190
U.S. Pat. No. 8,252,592
U.S. Pat. No. 8,339,645
U.S. Pat. No. 8,398,282
U.S. Pat. No. 7,446,179
U.S. Pat. No. 6,410,319
U.S. Pat. No. 7,070,995
U.S. Pat. No. 7,265,209
U.S. Pat. No. 7,354,762
U.S. Pat. No. 7,446,191
U.S. Pat. No. 8,324,353
U.S. Pat. No. 8,479,118
European patent application EP2537416
Sadelain et al., Cancer Discov. 2013 April; 3 (4): 388-398
Davila et al. (2013) PLOS ONE 8 (4): e61338
Turtle et al., Curr. Opin. Immunol., 2012 October; 24 (5): 633-39
Wu et al., Cancer, 2012 Mar. 18 (2): 160-75
Sambrook et al., Molecular Cloning: A Laboratory Manual, 3^{rd} ed., Cold Spring Harbor Press, Cold Spring Harbor, N.Y. 2001
Ausubel et al., Current Protocols in Molecular Biology, Greene Publishing Associates and John Wiley & Sons, NY, 1994

1. A method of protecting an implanted cell, tissue, organ or organoid from immune response in a recipient subject comprising administering to said recipient subject a chimeric antigen receptor (CAR) T regulatory cell (T_{reg}), wherein said CAR T_{reg} recognizes a natural or artificial antigen that (a) is not found in said implanted cell, tissue, organ or organoid and is (b) co-administered with said implanted cell, tissue, organ or organoid or found in proximity to said implanted cell, tissue, organ or organoid once implanted.

2. The method of claim 1, wherein said antigen is co-administered to said recipient subject and is associated with a particle (microparticle or nanoparticle), a polymer, a cell, a synthetic/engineered cell, or a synthetic cell surrogate.

3. The method of claim 1, wherein said antigen is found in proximity to said implanted cell, tissue, organ or organoid and located on an extracellular matrix or cell found at a site of implantation in said recipient subject.

4. The method of claim 1, wherein said CAR T_{reg} cells are generated from peripheral blood mononuclear cells.

5. The method of claim 1, wherein said CAR T_{reg} cells are generated from cells that are CD4+, CD25+, and CD127−.

6. The method of claim 5, wherein said CAR T_{reg} cells are generated from naïve cells that are CD4+, CD25+, CD127−, CD45RA+, and CD62L+.

7. The method of claim 1, wherein CAR T_{reg} cells are generated by anti-CD3/CD28 stimulation in the presence of IL-2, followed by transduction with a virus encoding the CAR, such as a lentivirus.

8. The method of claim 1, wherein CAR Treg cells are generated by anti-CD3/CD28 stimulation in the presence of IL-2, followed by CRISPR/Cas9 targeting of the alpha constant region of the T cell receptor (TCR) locus (TRAC) combined with transduction with a non-integrating adeno-associated virus (AAV) encoding the CAR.

9. The method of claim 7, wherein the transduced cells are expanded in the presence of IL-2.

10. The method of claim 1, wherein said CAR T_{reg} cells retain expression of FOXP3 and HELIOS at levels indicative of T_{reg} cells.

11. The method of claim 1, wherein the subject is a human or non-human mammal.

12. The method of claim 1, wherein the subject suffers from a disease or disorder benefiting from transplant therapy.

13. The method of claim 12, wherein the disease or disorder is cardiovascular disease, cancer, autoimmune disease (e.g., Type 1 diabetes, autoimmune carditis) or Parkinson's disease.

14. The method of claim 1, wherein said implanted cell, tissue, organ or organoid comprises cardiovascular cells, pancreatic beta cells, or dopaminergic neuron progenitors.

15. The method of claim 1, wherein said artificial antigen is a truncated CD19 antigen.

16. A method of transplanting a cell, tissue, organ, or organoid into a subject comprising:
(a) transplanting a cell, tissue, organ, or organoid into a subject; and
(b) introducing into the subject a chimeric antigen receptor (CAR) T regulatory cell (T_{reg}), wherein said CAR T_{reg} recognizes a natural or artificial antigen that (a) is not found in said implanted cell, tissue, organ or organoid and is (b) found in proximity to said implanted cell, tissue, organ or organoid once implanted.

17. The method of claim 16, wherein said subject suffers from a disease or disorder benefiting from transplant therapy.

18. A method of transplanting a cell, tissue, organ, or organoid into a subject comprising:
(a) transplanting a cell, tissue, organ or organoid into a subject; and
(b) administering to the subject (i) a chimeric antigen receptor (CAR) T regulatory cell (T_{reg}), wherein said CAR T_{reg} recognizes a natural or artificial antigen that is not found in said implanted cell, tissue, organ or organoid; and (ii) said natural or artificial antigen,
wherein said natural or artificial antigen co-administered with said CAR T_{reg} or implanted at or near the site at which said cell, tissue, organ, or organoid is implanted.

19. The method of claim 18, wherein said subject suffers from a disease or disorder benefiting from transplant therapy.

20. The method of claim 19, wherein the disease or disorder is cardiovascular disease, cancer, autoimmune disease (e.g., Type 1 diabetes, autoimmune myocarditis), or Parkinson's disease.

21. A kit comprising (a) a chimeric antigen receptor (CAR) T regulatory cell (T_{reg}) and (b) a natural or artificial antigen recognized by said CAR T_{reg}, optionally wherein said antigen is associated with a particle (microparticle or nanoparticle), a polymer, a cell, a synthetic cell, or a synthetic cell surrogate.

22. The kit of claim 21, further comprising (c) a functional organoid, such as a cardiac organoid.
